(19) United States
(12) Patent Application Publication
(10) Pub. No.: US 2007/0191861 A1
(43) Pub. Date: Aug. 16, 2007

(54) Instruments and methods for implanting nucleus replacement material in an intervertebral disc nucleus space

(75) Inventors: Randall N. Allard, Germantown, TN (US); Thomas J. Francis, Cardova, TN (US); Hai H. Trieu, Cordova, TN (US)

Correspondence Address: HESLIN ROTHENBERG FARLEY & MESITI P.C., 5 COLUMBIA CIRCLE, ALBANY, NY, 12203, US

(73) Assignee: SDGI Holdings, Inc., Wilmington, DE (US)

(21) Appl. No.: 11/343,088

(22) Filed: Jan. 30, 2006

(51) Int. Cl. A61F 2/00

(52) U.S. Cl. 606/99

(57) ABSTRACT

Instruments and methods are provided for implanting nucleus replacement material in an intervertebral disc nucleus space employing an inserter having proximal and distal end regions and a passageway effective for passing the material therethrough. The distal end region of the inserter includes a conical-shaped outer surface, and the passageway exits through a side surface of the inserter in the distal end region at other than the apex of the conical-shaped outer surface. The nucleus replacement material passing through the inserter exits into an intervertebral disc nucleus space when the passageway exit in the side surface at the distal end region is disposed in the nucleus space. The instrument may also include a cover locking mechanism having pivotally connected first and second cover plates with a latching tab and landing sized and configured to mate and lock the cover plates automatically when the inserter assumes an implanting configuration.

### CROSS-REFERENCE TO RELATED APPLICATIONS/PATENTS

This application contains subject matter which is related to the subject matter of the following applications/patents, which are hereby incorporated herein by reference in their entirety:

“Method and Apparatus for Delivering an Intervertebral Disc Implant”, Trieu et al., U.S. Ser. No. 10/717,687, filed Nov. 20, 2003, and published on Jun. 17, 2004 as Patent Application Publication No. US 2004/0117018 A1;

“Method and Apparatus for Delivering an Intervertebral Disc Implant”, Trieu et al., Patent Cooperation Treaty Application No. PCT/US2004/038750, and published on Jun. 9, 2005 as International Publication No. WO 2005/051246 A2;

“Intervertebral Disc Nucleus Implants and Methods”, Hai H. Trieu, U.S. Ser. No. 09/943,411, filed Aug. 30, 2001, and published on Feb. 28, 2002 as Patent Application Publication No. US 2002/0026244 A1;

“Intervertebral Disc Nucleus Implants and Methods” Hai H. Trieu, U.S. Ser. No. 10/459,630, filed Jun. 11, 2003, and published on Oct. 23, 2003 as Patent Application Publication No. US 2003/0199984 A1; and

“Intervertebral Disc Nucleus Implants and Methods”, Hai H. Trieu, U.S. Pat. No. 6,620,196 B1, issued Sep. 16, 2003.

### TECHNICAL FIELD

The present invention relates generally to instruments and methods for delivering a spinal implant, and more particularly, the instruments and methods for implanting material to augment, repair, or replace an intervertebral disc nucleus.

### BACKGROUND OF THE INVENTION

The human spine is a biomechanical structure with thirty-three vertebral members, and is responsible for protecting the spinal cord, nerve roots and internal organs of the thorax and abdomen. The spine also provides structure support for the body while permitting flexibility of motion. A significant portion of the population will experience back pain at some point in their lives resulting from a spinal condition. The pain may range from general discomfort to disabling pain that immobilizes the individual. Back pain may result from a trauma to the spine, be caused by the natural aging process, or may be the result of a degenerative disease or condition.

The intervertebral disc functions to stabilize the spine and to distribute forces between vertebral bodies. A normal disc includes a gelatinous nucleus pulposus, an annulus fibrosis and two vertebral end plates. The nucleus pulposus is surrounded and confined by the annulus fibrosis.

It is known that intervertebral discs are prone to injury and degeneration. For example, herniated discs are common, and typically occur when normal wear, or exceptional strain, causes a disc to rupture. Degenerative disc disease typically results from the normal aging process, in which the tissue gradually looses its natural water and elasticity, causing the degenerated disc to shrink and possibly rupture.

Intervertebral disc injuries and degeneration are frequently treated by replacing or augmenting the existing disc material. Current intervertebral disc replacement procedures tend to utilize full-sized implants, particularly hydrogels, to augment or replace the original disc nucleus. These materials are commonly implanted after first making a hole with a guide wire, and then subsequently enlarging the hole with a succession of sleeves having increased diameters. Alternatively, a larger hole may be made by surgical incision, using a scalpel or a small diameter coring blade.

One problem associated with such implants is that they require a relatively large hole to be cut in the disc annulus to allow introduction of the implant. Since the hole must be large enough to accommodate a full sized implant, the annulus must be plugged or sewn closed after implantation to avoid allowing the implant to be expelled from the disc. This complicates the procedure, adding surgical time and cost, and leaving a less sound annulus when the procedure is complete.

Moreover, the devices heretofore used to deliver a spinal disc implant have been difficult to load and operate.

A need therefore remains for further instruments and methods of implanting spinal disc implants, and particularly for instruments and methods that avoid the need to make large incisions in the disc annulus, and are easy to load. The instruments and methods disclosed herein address these needs.

### SUMMARY OF THE INVENTION

The shortcomings of the prior art are overcome and additional advantages are provided, in one aspect, through provision of an instrument for implanting nucleus replacement material into an intervertebral disc nucleus space. The instrument includes an inserter comprising a passageway effective for passing nucleus replacement material therethrough. The inserter has a proximal end region and a distal end region. The distal end region of the inserter includes a conical-shaped outer surface, and the passageway exits through a side surface of the inserter in the distal end region thereof at other than the apex of the conical-shaped outer surface. The nucleus replacement material passing through the inserter can exit the passageway into an intervertebral disc space when the passageway exit in the side surface of the inserter at the distal end region thereof is at least partially disposed in the intervertebral disc nucleus space.

In another aspect, an instrument for implanting a nucleus replacement disc is presented which includes a first channel member and a second channel member. The first channel member has a first end region and a second end region, and defines a first passageway from the first end region to the second end region thereof. The first passageway is defined by at least one sidewall of the first channel member. A first post, located at the first end region of the first channel member, extends radially inward from the at least one sidewall of the first channel member. The second channel member includes a first end region and a second end region. The second channel member includes at least one sidewall defining a second passageway from the first end region to the second end region thereof. A second post, disposed at the first end region of the second channel member, extends radially inward from the at least one sidewall of the second channel member. The first channel member and the second channel member are pivotally connected at their respective first end regions. The instrument assumes a loading configuration when the first channel member and the second channel member are pivoted to define an angle less than 180 degrees, and assumes an implanting configuration when the first channel member and the second channel member are pivoted to define an angle of approximately 180 degrees. In the implanting configuration, the first passageway and the second passageway align to form a single aligned passageway. The second end region of the first channel member has a conical-shaped outer surface. The aligned passageway exits through a side surface of the first channel member at the first end region thereof at other than the apex of the conical-shaped outer surface. A nucleus replacement disc passing through the aligned passageway exits into an intervertebral disc nucleus space when the passageway exit at the first end region of the first channel member is at least partially disposed in the intervertebral disc nucleus space.

In yet another aspect, an instrument for implanting nucleus replacement material is presented. This instrument includes an inserter defining a passageway effective for passing nucleus replacement material therethrough. The inserter has a proximal end region and a distal end region, and includes a first channel member and a second channel member pivotally connected together at respective first ends thereof. The inserter assumes a loading configuration when the first channel member and the second channel member are pivoted to define an angle of less than 180 degrees, and assumes an implanting configuration when the first channel member and the second channel member are pivoted to define an angle of approximately 180 degrees. The first and second channel members define the passageway when the inserter is in the implanting configuration. The instrument further includes a cover locking mechanism including a first cover plate and a second cover plate. The first cover plate includes a proximal end and a distal end. The distal end of the first cover plate is pivotally connected to the first channel member near the first end thereof. The second cover plate also includes a proximal end and a distal end. The proximal end of the second cover plate is pivotally connected to the second channel member near the first end thereof, and the distal end of the second cover plate is pivotally connected to the first cover plate intermediate the proximal and distal ends of the first cover plate. The first cover plate further includes a latching tab at the proximal end thereof. The second cover plate is configured with a tab receiving landing. The latching tab and the tab receiving landing are configured to mate and lock the first cover plate and the second cover plate together as the inserter assumes the implanting configuration.

In a further aspect, a method of implanting material in an intervertebral disc nucleus space is provided. The method includes: providing a disc nucleus implant instrument having an inserter including a passageway effective for passing a material for replacing or augmenting an intervertebral disc nucleus, the inserter having a proximal end region and a distal end region, and wherein the distal end region of the inserter comprises a conical-shaped outer surface, and wherein the passageway exits through a side surface of the inserter in the distal end region thereof at other than the apex of the conical-shaped outer surface, the distal end region of the inserter with the conical-shaped outer surface being sized to function as a dilator; providing a material suitable for replacing or augmenting an intervertebral disc nucleus in the passageway of the inserter; providing a hole in the annulus of a disc receiving the material for replacing or augmenting an intervertebral disc nucleus, the hole having an undilated size that is smaller than the cross-sectional size of the material for replacing or augmenting the intervertebral disc nucleus, the hole having a dilated size that is larger than the cross-sectional size of the material for replacing or augmenting the intervertebral disc nucleus; introducing the dilating distal end region of the inserter into the hole in the disc annulus to dilate the hole in the disc annulus, the introducing comprising positioning the passageway exit at the distal end region of the inserter at least partially within the intervertebral disc nucleus space; passing the material for replacing or augmenting the intervertebral disc nucleus through the passageway exit in the side surface of the inserter in the distal end region; and withdrawing the inserter, and allowing the hole in the disc annulus to return to a smaller size than its dilated size.

In a still further aspect, a method of implanting material into an intervertebral disc nucleus space is provided. This method includes providing a disc nucleus implant instrument comprising an inserter and a cover locking mechanism. The inserter includes a passageway effective for passing material for replacing or augmenting an intervertebral disc nucleus, and includes a proximal end region and a distal end region, along with a first channel member and a second channel member pivotally connected together at respective first ends thereof. The inserter assumes a loading configuration when the first channel member and the second channel member are pivoted to define an angle of less than 180 degrees, and assumes an implanting configuration when the first channel member and the second channel member are pivoted to define an angle of approximately 180 degrees. The first and second channel members define the passageway when the inserter is in the implanting configuration. The cover locking mechanism includes a first cover plate and a second cover plate. The first cover plate includes a proximal end and a distal end, with the distal end of the first cover plate being pivotally connected to the first channel member near the first end thereof. The second cover plate includes a proximal end and a distal end, with the proximal end of the second cover plate being pivotally connected to the second channel member near the first end thereof, and the distal end of the second cover plate being pivotally connected to the first cover plate intermediate the proximal and distal ends of the first cover plate. The first cover plate is configured with a latching tab at the proximal end thereof, and the second cover plate is configured with a tab receiving landing. The latching tab and the tab receiving landing are sized and configured to mate and lock the first cover plate and second cover plate together as the inserter assumes the implanting configuration.

The method further includes: providing a material suitable for replacing or augmenting an intervertebral disc nucleus in the passageway of the inserter while the inserter is in the loading configuration; transitioning the inserter from the loading configuration to the implanting configuration, the transitioning including pivoting the first channel member and the second channel member to define an angle of approximately 180 degrees, and simultaneous therewith, pivoting the first cover plate towards the second cover plate so that the latching tab of the first cover plate mates and locks with the tab receiving landing of the second cover plate; providing a hole in the annulus of a disc receiving the material for replacing or augmenting an intervertebral disc nucleus; introducing the distal end region of the inserter into the hole in the disc annulus, the introducing including positioning the distal end region of the inserter within the intervertebral disc nucleus space; passing the material for replacing or augmenting the intervertebral disc nucleus through a passageway exit in the distal end region of the inserter; and withdrawing the distal end region of the inserter from the intervertebral disc nucleus space.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1 illustrates one embodiment of a prosthetic nucleus replacement disc to be implanted into an intervertebral disc nucleus space of a patient, in accordance with an aspect of the present invention;

FIG. 2 illustrates the nucleus replacement disc of FIG. 1 in a partially straightened configuration, in accordance with an aspect of the present invention;

FIG. 3 illustrates the nucleus replacement disc of FIGS. 1 & 2 in a nearly straightened configuration, in accordance with an aspect of the present invention;

FIG. 4 is a schematic of one embodiment of an inserter (shown in a loading configuration) for facilitating placement of a nucleus replacement disc in the intervertebral disc nucleus space of a patient, in accordance with an aspect of the present invention;

FIG. 5 is an isometric view of another embodiment of an inserter (shown in implanting configuration) for implanting nucleus replacement material into an intervertebral disc nucleus space of a patient, in accordance with an aspect of the present invention;

FIG. 6 is a cross-sectional elevational view of the inserter of FIG. 5, in accordance with an aspect of the present invention;

FIG. 7 is a partially enlarged, isometric view of the inserter of FIGS. 5 & 6, showing the inserter transitioning from a loading configuration to an implanting configuration, and illustrating one embodiment of a cover locking mechanism, in accordance with an aspect of the present invention;

FIG. 8 is a partial elevational view of the inserter of FIGS. 5-7, showing the nucleus replacement disc of FIGS. 1-3 loaded therein, in accordance with an aspect of the present invention;

FIG. 9 is an isometric view of one embodiment of a handle assembly configured to releasably engage the inserter of FIGS. 5-6, in accordance with an aspect of the present invention;

FIG. 10 is a partial cross-sectional elevational view of the handle assembly of FIG. 9, in accordance with an aspect of the present invention;

FIG. 10A is a partially enlarged view of certain handle assembly components of FIG. 10, in accordance with an aspect of the present invention;

FIG. 11 is a partial isometric view of the handle assembly of FIG. 9 releasably engaging the inserter of FIGS. 5 & 6, and illustrating a spring-biased release mechanism, in accordance with an aspect of the present invention;

FIG. 12 is an isometric view of one embodiment of an assembled implanting instrument, with the inserter of FIGS. 5 & 6 releasably engaged in the handle assembly of FIG. 9, and illustrating assembly and operation of the instrument, in accordance with an aspect of the present invention;

FIG. 13 is a schematic view of the instrument of FIG. 12 showing passing of a nucleus replacement disc through the passageway within the inserter, in accordance with an aspect of the present invention; and

FIG. 14 is a partial isometric view of the instrument of FIG. 13, showing the nucleus replacement disc exiting the inserter through the side surface opening therein for positioning within an intervertebral disc nucleus space, in accordance with an aspect of the present invention.

### BEST MODE FOR CARRYING OUT THE INVENTION

As indicated above, the present invention provides in one aspect, an improved method and instrument for implanting a prosthetic disc nucleus. In one embodiment, the method comprises: (a) providing a disc nucleus implant instrument having: (i) a lumen or passageway effective for passing a material for augmenting, repairing, or replacing an intervertebral disc nucleus, the passageway having a proximal end and a distal end; and (ii) a dilator at the distal end of the passageway, the dilator comprising a conical-shaped outer surface effective for dilating an opening in a disc annulus, and a passageway exit in a side surface of the inserter at other than the apex of the conical-shaped outer surface; (b) providing a disc nucleus material in the passageway of the disc nucleus implant instrument, the disc nucleus material being suitable for augmenting, repairing, or replacing an intervertebral disc nucleus, and the disc nucleus material having a first cross-sectional size; (c) providing a hole in the annulus of an intervertebral disc, the hole having an undilated size that is smaller than the first cross-sectional size of the disc nucleus material, and the hole having a dilated size that is larger than the first cross-sectional size of the disc nucleus material; (d) introducing the dilator of the disc nucleus implant instrument into the hole in the disc annulus while the hole is not fully dilated, thereby causing the hole to dilate; (e) passing the disc nucleus material through the dilator and into the disc nucleus space while the hole in the disc annulus is dilated; and (f) withdrawing the disc nucleus implant instrument and allowing the hole in the disc annulus to return to a size smaller than its dilated size.

It is to be appreciated from the above description that the inventive method finds utility with any material effective for augmenting, repairing, or replacing an intervertebral disc nucleus. Some materials effective for that purpose are described in greater detail in U.S. patent application Ser. No. 10/245,955 (published Mar. 18, 2004 as Patent Application Publication No. US 2004/0054414 A1), in U.S. patent application Ser. No. 10/645,006 (published Feb. 24, 2005 as Patent Application Publication No. US 2005/0043801 A1), in U.S. patent application Ser. No. 10/426,613 (published Nov. 4, 2004 as Patent Application Publication No. US 2004/0220631 A1), and in U.S. patent application Ser. No. 60/411,514, all of which are incorporated herein by reference in their entirety. Other materials are known to persons skilled in the art, or can be identified without undue experimentation.

The materials implanted by the inventive methods are referred to herein as “disc nucleus material” or “nucleus replacement material” since they will typically be used as such, even though the material may not come from a disc nucleus. Accordingly, as used herein, a disc nucleus material or a nucleus replacement material is any material that is to be used to augment, repair, or replace all or a portion of an intervertebral disc nucleus in the context of this application, regardless of the source of that material.

When a material effective for augmenting, repairing, or replacing an intervertebral disc nucleus has been identified, it is implanted in the disc nucleus space of the disc being repaired. Various instruments designed for that purpose, referred to herein as a disc nucleus implant instrument, may be used.

As indicated above, the disc nucleus implant instrument includes an inserter with a passageway effective for passing the disc nucleus material into an intervertebral disc nucleus. The inserter has a proximal end and a distal end. The passageway is sized and configured to allow passage of the disc nucleus material and is accordingly (in one embodiment) straight and smooth on its interior surface. Ridges, indentations, projections, etc., may be provided on the interior surface to the extent they assist in, or do not prevent, passage of the material through the passageway.

By way of example only, the instruments disclosed herein may include a passageway having an inner diameter of between about 2 mm to about 20 mm, with an inner diameter of between about 5 mm and about 10 mm being preferred in one embodiment. The length of the passageway is between about 5 cm and about 30 cm, with a length of between about 10 cm and about 25 cm being preferred in one embodiment.

The disc nucleus implant instrument is also configured with a dilator at the distal end of the inserter through which the passageway exits. The dilator is designed to be effective for dilating a small opening in a disc annulus so that the opening is made large enough for the inserter containing the material being implanted to pass through. The dilator dilates the opening without tearing the annulus, so that the dilated opening shrinks back to a smaller size after the disc nucleus implant instrument is removed. In one embodiment described hereinbelow, the dilator is the distal end region of the inserter, and includes a conical-shaped outer surface wherein the passageway exits through a side surface of the inserter in the distal end region at other than the apex of the conical-shaped outer surface, leaving a solid apex surface at the tip of the distal end region.

The apex of the conical-shaped outer surface at the tip of the distal end region of the inserter is non-pointed, and more particularly, either blunt or rounded, to minimize the possibility of puncturing or penetrating the anterior annulus during dilation and insertion of the instrument, or during delivery of the disc nucleus material. By way of specific example, a radius of a rounded apex, in accordance with an aspect of the present invention, is not less than 0.5 mm, and preferably not less than 1 mm.

To implant material, a small incision (preferably a hole) is first cut in the annulus of the disc being repaired or augmented. A guide wire or other small instrument may be used to make the initial hole. If necessary, successively larger holes are cut from an initially small puncture. The purpose of the hole (also called an aperture, an opening, or a portal, for example) is to allow passage of the distal end of the inserter with new disc nucleus material, so that the material can be implanted into the disc nucleus space from the side (i.e., through the annulus). It is important, though, for the hole to be as small as possible to minimize expulsion of the material through the hole after the surgery is complete.

Once a small hole is provided, the conical-shaped outer surface of the disc nucleus implant instrument's inserter is inserted into the hole. The conical-shaped end dilates the hole, making it large enough to position the distal end of the inserter in the interbody space and deliver the material being used to replace or augment the disc nucleus. The dilator preferably stretches the hole temporarily, and avoids tearing so that the hole can return back to its undilated size after the instrument is removed. Even if some tearing or permanent stretching occurs, the dilation is preferably accomplished in a manner that allows the hole to return to a size smaller than its dilated size after the surgery is complete.

The material being used to replace or augment the disc nucleus is then implanted into the disc nucleus space, typically by forcing it through the passageway of the instrument, through the sidewall exit, and into the disc nucleus space. The tip of the instrument may be moved from side-to-side, or from front-to-back, as necessary to deliver the material uniformly throughout the disc nucleus space.

After the material is delivered into the disc nucleus space, the instrument is withdrawn and the hole in the annulus is allowed to return to its original size. If the annulus has been stretched or torn so that it does not return to its original size, it should at least return to a size smaller than its dilated size.

In one embodiment, the method described above is used to deliver a material that has two configurations—a first configuration and a second configuration—wherein one of the configurations presents a cross-section that is smaller than the other configuration. With this embodiment, it is possible to implant the material through the dilated annular opening when the material is it its smaller configuration, and then cause or allow the material to assume its larger configuration after it has been passed through the inserter positioned in the dilated hole in the annulus.

Briefly summarizing this aspect of the invention, one embodiment comprises: (a) providing a disc nucleus implant instrument having: (i) an inserter with a passageway for passing a prosthetic disc nucleus, the passageway having a proximal end and a distal end; and (ii) a dilator at the distal end of the passageway, the dilator comprising a conical-shaped outer surface effective for dilating an opening in a disc annulus, and a passageway exit in a side surface of the inserter at other than the apex of the conical-shaped outer surface; (b) providing a prosthetic disc nucleus having a first configuration and a second configuration, wherein the first configuration presents a first cross-sectional size and the second configuration presents a second cross-sectional size, wherein the first cross-sectional size is larger than the second cross-sectional size; (c) providing a hole in the annulus of a disc receiving the prosthetic disc nucleus, the hole having an undilated size that is smaller than the first cross-sectional size of the prosthetic disc nucleus, and the hole having a dilated size that is larger than the second cross-sectional size of the prosthetic disc nucleus; (d) providing the prosthetic disc nucleus in its second configuration in the passageway of the disc nucleus implant instrument; (e) introducing the dilator of the disc nucleus implant instrument into the hole in the disc annulus while the hole is not fully dilated; (f) passing the prosthetic disc nucleus through the dilator and into the disc nucleus space while the disc annulus is more fully dilated with the inserter disposed therein and the prosthetic disc nucleus is in its second configuration; (g) withdrawing the disc nucleus implant instrument and allowing the disc annulus to return to a size smaller than its dilated size; and (h) causing or allowing the prosthetic disc nucleus to assume its first configuration.

It is to be appreciated that the inventive method described above finds particular utility with materials described in U.S. patent application Ser. No. 10/645,006 (Patent Application Publication No. US 2005/0043801 A1), and in U.S. patent application Ser. No. 10/426,613 (Patent Application Publication No. US 2004/0220631 A1). Both of those applications disclose materials that may be dehydrated prior to implantation, and are then rehydrated to a larger size after implantation. The inventive methods described above also find particular utility with materials described in the above-incorporated U.S. patent application Ser. No. 09/943,441 (Patent Application Publication No. US 2002/0026244 A1), which discloses implants having a shape memory that allows the implant to be straightened to a straightened configuration having a smaller cross-section before implantation, and then relaxed to a folded configuration having a larger cross-section after implantation.

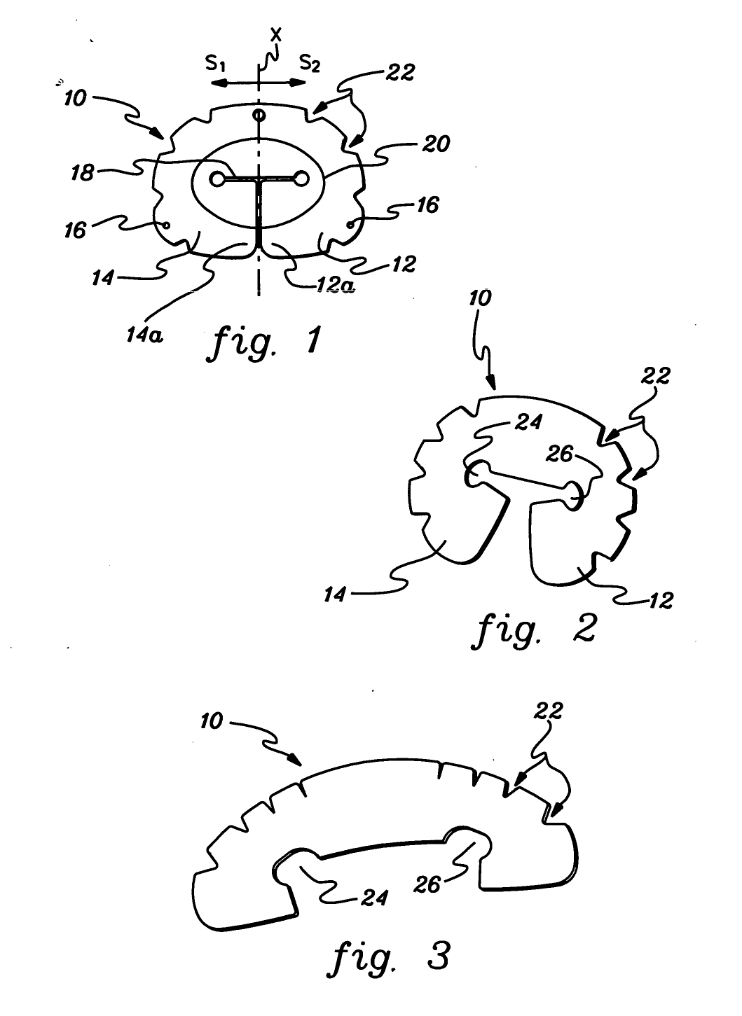

For example, FIGS. 1-3 (discussed in more detail below) show one embodiment of an implant that may be used in the present invention. Referring to these figures, implant 10 comprises a folded implant having shape memory so that it can be unfolded for implantation, yet return to its folded configuration when relaxed in the disc nucleus space. As described in U.S. patent application Ser. No. 09/942,411, implant 10 has two arms 12 and 14 that are folded over to create inner fold 18. The arms preferably abut one another at their ends when in the folded configuration, and also abut the middle portion of the implant. This creates an implant having a substantially sold center core, and provides the support necessary to avoid compression of the disc nucleus in most patients.

Additionally, the illustrated implant may have external side surfaces that include at least one groove extending along the surface to advantageously further relieve the compressive force on the external side of the implant when the implant is deformed into a substantially straightened, or otherwise unfolded configuration. This allows extensive short-term deformation without permanent deformation, cracks, tears or other breakage. For example, implant 10 shown in FIGS. 1-3, includes a plurality of grooves 22 disposed along its external surface, with the grooves typically extending from the top surface to the bottom surface of the implant. When dividing the implant in half, thus more easily viewing a first side S₁ and a second side S₂, with a plane passing through the width of the implant along axis X, it can be seen in FIG. 1 that four grooves are present on first side S₁ and four grooves are present on second side S₂, although more or less may be present depending on the case. It is preferred that at least one groove is present on each side S₁ and S₂.

As to the specifics of the method used to deliver the “two configuration” implants, the basic principles of cutting a small hole in the disc annulus and dilating the annulus enough to allow the disc nucleus material to pass through the hole apply. In this embodiment though, when the instrument is withdrawn the material is caused or allowed to assume a shape and/or size larger than the shape/size that was presented when the material was implanted. For example, when a dehydrated material is used, the material is allowed to swell up in the disc space so that the rehydrated material is larger than the dehydrated material. Then, when the instrument is withdrawn and the hole in the annulus returns to a smaller size, the disc nucleus material finds it even more difficult to fit back through the hole. This further mitigates the need for an annular plug or sutures to prevent expulsion of the disc nucleus material.

When shape memory implants such as those discussed in U.S. patent application Ser. No. 09/943,411 are being used, the method may include the step of unfolding the implant so that it assumes a “straightened” configuration in the delivery instrument. The implant may then be delivered via the inserter through the dilated hole while in that straightened configuration. After implantation, the implant returns naturally to its relaxed, folded configuration that mimics the shape of a natural disc. In this folded configuration the implant is too large to easily fit back through the undilated hole.

One disc nucleus implant instrument is next described that may be used (in accordance with an aspect of the present invention) to deliver one embodiment of a “two configuration” disc nucleus material. In one embodiment the instrument includes: (a) an inserter having a proximal end and a distal end; (b) means for converting a disc nucleus implant from a first, folded configuration to a second, straightened configuration; (c) means for positioning the disc nucleus implant in the channel member while the disc nucleus implant is in its second straightened configuration; and (d) means for moving the disc nucleus implant through the channel and into an intervertebral disc space while the implant remains substantially in its straightened configuration.

More specifically, in one embodiment the instrument includes: (a) a first channel member having a first end region and a second end region, the first channel member defining a passageway between the first end region and the second end region, and including at least one sidewall; (b) a first post extending radially inward from the first channel member sidewall, the first post being located near the first end region of the first channel member; (c) a second channel member having a first end region and a second end region, the second channel member defining a passageway from the first end region to the second end region thereof, and comprising at least one sidewall; (d) a second post extending radially inward from the second channel member sidewall, the second post being located near the first end region of the second channel member; wherein the first channel member and the second channel member are pivotally connected at their respective first end regions; wherein the instrument assumes a loading configuration when the first channel member and the second channel member are pivoted to define an angle of less than 180 degrees, and assumes an implanting configuration when the first channel member and the second channel member arc pivoted to define an angle of approximately 180 degrees.

In other embodiments the instrument may include a cover locking mechanism to lock the instrument in its implanting configuration. As will be described further below, the cover locking mechanism automatically locks the instrument in a manner that maintains the angle of approximately 180 degrees as the instrument assumes the implanting configuration, thereby keeping the implant in its straightened configuration and thus facilitating implantation.

When an instrument having a dilator is used to implant a shape memory implant as described above, one aspect of the present invention provides a method comprising: (a) providing a disc nucleus implant instrument including: (i) a first channel member having a first end region and a second end region, the first channel member defining a passageway from the first end region to the second end region, and including at least one sidewall; (ii) a first post extending radially inward from the first channel member sidewall, the first post being located near the first end of the first channel member; (iii) a second channel member having a first end region and a second end region, the second channel member defining a passageway from the first end region to the second end region, and including at least one sidewall; and (iv) a second post extending radially inward from the second channel member sidewall, the second post being located near the first end region of the second channel member; wherein the first channel member and the second channel member are pivotally connected at their respective first ends; wherein the instrument assumes a loading configuration when the first channel member and the second channel member are pivoted to define an angle of less than 180 degrees, and assumes an implanting configuration when the first channel member and the second channel member are pivoted to define an angle of approximately 180 degrees; (b) providing a prosthetic disc nucleus comprising a load bearing elastic body having shape memory and sized for placement into an intervertebral disc space, the body having a first end, a second end, and a central portion; wherein the shape memory biases the body to a first configuration wherein the first end and the second end are positioned adjacent to the central portion to form at least one inner fold and to provide a substantially solid center core when the implant is in its first configuration; the elastic body being configurable into a second, straightened configuration for insertion through an opening in an intervertebral disc annulus fibrosis; wherein the shape memory returns the body to the first configuration after the insertion; wherein the prosthetic disc nucleus presents a first cross-sectional size when in its first configuration, and a second cross-sectional size when in its second configuration, wherein the first cross-sectional size is larger than the second cross-sectional size; (c) loading the prosthetic disc nucleus implant instrument such that the first post extending radially inward from the first channel member sidewall and the second post extending radially inward from the second channel member sidewall each are positioned in the inner fold of the prosthetic disc nucleus; (d) converting the disc nucleus implant instrument from its loading configuration to its implanting configuration, thereby moving the first post and the second post further apart and straightening the prosthetic disc nucleus from its first configuration to its second configuration; (e) providing a hole in the annulus of a disc receiving the prosthetic disc nucleus, the hole having an undilated size that is smaller than the first cross-sectional size of the prosthetic disc nucleus, and the hole having a dilated size that is larger than the second cross-sectional size of the prosthetic disc nucleus; (f) introducing the dilator of the disc nucleus implant instrument into the hole in the disc annulus, thereby dilating the opening; (g) passing the prosthetic disc nucleus through the passageway exit and into the disc nucleus space while the disc annulus is dilated and the prosthetic disc nucleus is in its second configuration; (i) withdrawing the disc nucleus implant instrument and allowing the disc annulus to return to a size smaller than its dilated size; and (j) causing or allowing the prosthetic disc nucleus to assume its first configuration.

As to other disc nucleus implants that may be used in the present invention, some spinal disc implants comprise a biomechanical or otherwise flexible material to facilitate its conversion from a loading configuration to a deliverable configuration. Further, the spinal disc implant may include a load bearing elastic body surrounded by an outer, resorbable or otherwise temporary, shell. The outer shell advantageously anchors the elastic body within the intervertebral disc space. The surface of the implant may include various surface features, including various macro-surface patterns, and chemical or physical modifications to further enhance fixation of the implant. The surface features, such as the macro-surface patterns and physical modifications, for example, may enhance fixation of the elastic body to the outer shell, or they may enhance fixation to surrounding tissue such that, in certain forms of the invention, no outer shell is needed.

The dimensions of the spinal disc implants used herein may vary depending on the particular case, but the implant is typically sized for introduction into an intervertebral disc nucleus space. Moreover, the implant is preferably wide enough to support adjacent vertebrae and is of height sufficient to separate the adjacent vertebrae.

The spinal disc implants used in the invention may be fabricated in a wide variety of shapes, as desired for a particular application. Although the implant may assume a variety of shapes, it is typically shaped to conform to the shape of the natural nucleus pulposus, at least when in its hydrated and/or relaxed configuration. Thus, the implants may be substantially elliptical when in their hydrated and/or relaxed configurations. In other forms of the invention, the shape of the implants in their hydrated and/or relaxed configurations may be generally annular-shaped, cylindrical-shaped, or otherwise shaped as required to conform to the intervertebral disc cavity.

The spinal disc implants are also shaped in a manner to allow easy implantation into a spinal disc nucleus space. Accordingly, the implant may have a narrow, tubular shape when in its dehydrated and/or straightened configuration, and may include at least one narrow or pointed end to facilitate implantation through a small annulus hole.

Although the implants may be formed as a one-piece implant, it may also be formed as a multi-piece implant. When one-piece implants are used, they may be used individually or they may be used in a combination of two or more implants. When multi-piece implants are used, the pieces may be used independently or they may be joined together. In some embodiments, one-piece implants and multi-piece implants are used together.

A spinal disc implant for use in the invention may be formed from a wide variety of biocompatible polymeric materials, including elastic materials, such as elastomeric materials, hydrogels or other hydrophilic polymers, or composites thereof. Suitable elastomers include silicone, polyurethane, copolymers of silicone and polyurethane, polyolefins, such as polyisobutylene and polyisoprene, neoprene, nitrile, vulcanized rubber and combinations thereof. The vulcanized rubber described herein may be produced, for example, by a vulcanization process utilizing a copolymer produced as described, for example, in U.S. Pat. No. 5,245,098 to Summers et al. from 1-hexene and 5-methyl-1,4-hexadiene. Suitable hydrogels include natural hydrogels, and those formed from polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrrolidone, N-vinyl lactams, acrylamide, polyurethanes and polyacrylonitrile, or may be other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyetherurethane. Other suitable hydrophilic polymers include naturally occurring materials such as glucomannan gel, hyaluronic acid, polysaccharides, such as cross-linked carboxyl-containing polysaccharides, and combinations thereof. The nature of the materials employed to form the elastic body should be selected so the formed implants have sufficient load bearing capacity. In certain embodiments, a compressive strength of at least about 0.1 Mpa is desired, although compressive strengths in the range of about 1 Mpa to about 20 Mpa may also be preferred.

When the implants are formed from elastic materials, such as hydrogel, or other similar hydrophilic material, or include a resorbable outer shell, they may advantageously deliver desired pharmacological agents. The pharmacological agent may be a growth factor that may advantageously repair the endplates and/or the annulus fibrosis. For example, the growth factor may include an osteoinductive factor (e.g., a bone morphogenetic protein), transforming growth factor-ss (TGF-ss), insulin-like growth factor, platelet derived growth factor, fibroblast growth factor or other similar growth factor or combination thereof having the ability to repair the endplates and/or the annulus fibrosis of an intervertebral disc. In one embodiment, the spinal disc implant comprises an osteoinductive factor.

Osteoinductive factors can be defined as those factors, which stimulate uncommitted cells, e.g., mesenchymal stem cells, to convert phenotypically to chondroprogenitor and osteoprogenitor cells. Osteogenic factors include those factors that contain cells that are committed to osteoblastic phenotypes or stimulate committed osteoprogenitor cells and mature osteoblasts to proliferate. Thus, the major distinction between the two factors is that cellular proliferation characterizes an osteogenic factor, whereas cellular differentiation characterizes an osteoinductive factor. It will be understood that an osteoinductive factor and an osteogenic factor can be contained in a spinal disc implant either alone, or in combination, providing for a synergistic effect.

Suitable osteoinductive factors for use in the invention include growth factors to stimulate or induce bone growth, including factors comprised of protein or genes. Recombinant human bone morphogenetic proteins (rhBMPs) are preferred. More particularly, the bone morphogenetic protein may be a rhBNMP-2, rhBMP-4 or heterodimers thereof. Bone morphogenic protein (BMP), an osteoinductive cytokine extracted from bone matrix, is capable of inducing bone formation when implanted in a fracture of surgical bone site. BMP actually refers to a group of bone morphogenic proteins belonging to the TGF-β superfamily. The structures of eleven proteins, BMP-1 through BMP-13 have been elucidated. Recombinantly produced human bone morphogenic protein-2 has been demonstrated in several animal models to be effective in regenerating bone in skeletal defects.

Recombinant BMP-2 can be used at a concentration of about 0.4 mg/ml to about 4.0 mg/ml, preferably about 1.0 to 3.0 mg/ml. However, any bone morphogenetic protein is contemplated including bone morphogenetic proteins designated as BMP-1 through BMP-13. BMPs are available from Genetics Institute, Inc., Cambridge, Mass. and may also be prepared by one skilled in the art as described in U.S. Pat. No. 5,187,076 to Wozney et al.; U.S. Pat. No. 5,366,875 to Wozney et al.; U.S. Pat. No. 4,877,864 to Wang et al.; U.S. Pat. No. 5,108,922 to Wang et al.; U.S. Pat. No. 5,116,738 to Wang et al.; U.S. Pat. No. 5,013,649 to Wang et al.; U.S. Pat. No. 5,106,748 to Wozney et al.; and PCT Patent Nos. WO93/00432 to Wozney et al.; WO94/26893 to Celeste et al.; and WO94/26892 to Celeste et al. All osteoinductive factors are contemplated whether obtained as above or isolated from bone. Methods for isolating bone morphogenetic protein from bone are described in U.S. Pat. No. 4,294,753 to Urist, and Urist et al., 81 PNAS 371, 1984.

In other forms of the invention, the spinal disc implants may comprise a pharmacological agent used for treating various spinal conditions, including degenerative disc disease, spinal arthritis, spinal infection, spinal tumor and osteoporosis.

Such agents include antibiotics, analgesics, anti-inflammatory drugs, including steroids, and combinations thereof. Other such agents are well known to the skilled artisan. These agents are also used in therapeutically effective amounts. Such amounts may be determined by the skilled artisan depending on the specific case.

The pharmacological agents are preferably dispersed within the hydrogel, or other hydrophilic, implant for in vivo release, and/or, with respect to the implants with the resorbable other shell, may be dispersed in the outer shell. The hydrogel can be cross-linked chemically, physically, or by a combination thereof, in order to achieve the appropriate level of porosity to release the pharmacological agents at a desired rate. The agents may be released upon cyclic loading, and, in the case of implants including a resorbable outer shell, upon resorption of the shell.

The pharmacological agents may be dispersed in the implants by adding the agents to the solution used to form the implant, by soaking the formed implant in an appropriate solution containing the agent, or by other appropriate methods known to the skilled artisan. In other forms of the invention, the pharmacological agents may be chemically or otherwise associated with the implant. For example, the agents may be chemically attached to the outer surface of the implant.

Referring further to the drawings, and as noted briefly above, FIGS. 1-3 depict one embodiment of a disc nucleus implant, generally denoted 10, that may be implanted with a disc delivery instrument such as described hereinbelow. Implant 10 comprises a pair of arms 12 & 14 that are folded to form an inner fold 18 when the implant is in its relaxed configuration (see FIG. 1). The folded arms abut one another at their ends 12A & 14A when the implant is relaxed, so that the center core 20 of the implant is substantially solid.

Apertures 24 & 26 are provided to correspond to posts of the disc delivery instrument (described below). When the posts are inserted into the apertures and the hinged channel members are pivoted to an angle of about 180 degrees, implant 10 straightens to provide a cross-sectional size that is less than the cross-sectional size of the folded implant. Grooves 22 are provided on the outer surface to prevent cracking or tearing of the implant when the implant is in its straightened configuration. X-ray markers, such as tantalum markers 16 may be included to assist in positioning the implant. A larger x-ray marker may be provided in the anterior portion of the implant, and smaller x-ray markers provided in the posterior portions of the implant. It will be appreciated that the methods and instruments disclosed herein may be used with a surgical approach that is posterior, anterior, lateral or oblique.

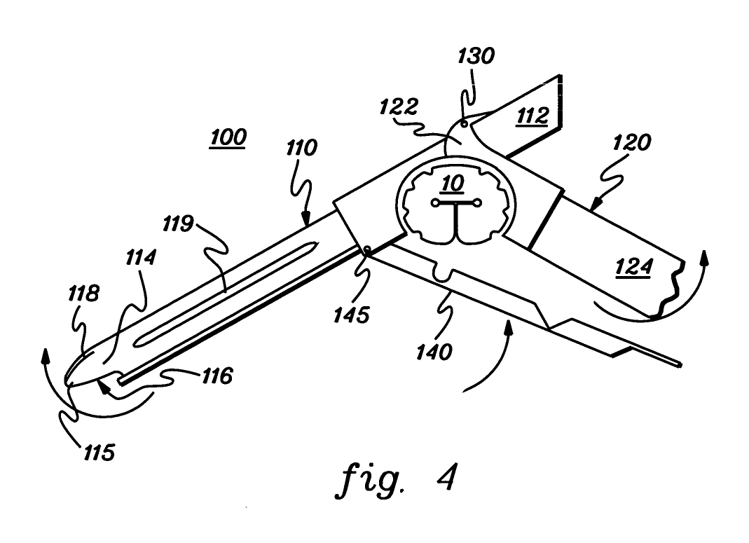

In the various instrument embodiments depicted herein, the instrument includes an inserter and a handle assembly. FIG. 4 and FIGS. 5-8 depict different embodiments of an inserter. With each embodiment of the instrument, different sized inserters may be provided corresponding to specific implant sizes. Each inserter interfaces with a handle assembly and pusher rod, one embodiment of which is described below with reference to FIGS. 9-11. FIGS. 12-14 illustrate one assembled embodiment of an implanting instrument, in accordance with an aspect of the present invention, and show use of the implanting instrument in delivering nucleus replacement material into an intervertebral disc nucleus space.

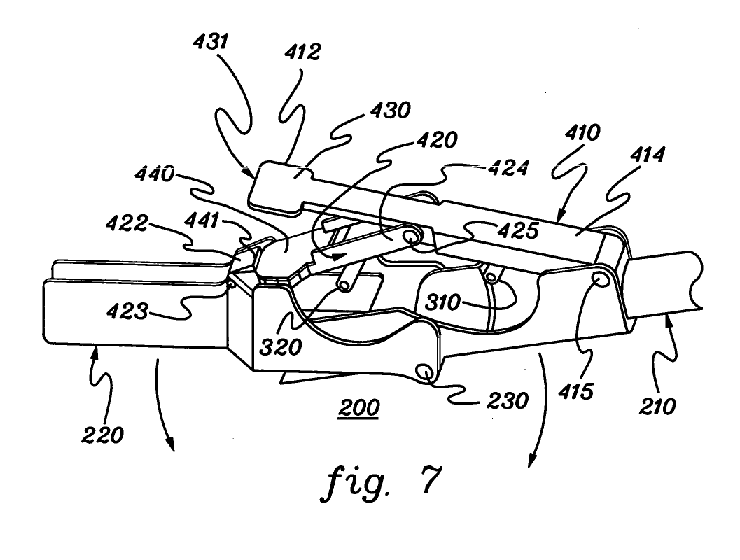

Referring to FIG. 4, one embodiment of an inserter 100 is shown, in accordance with aspects of the present invention. Inserter 100 includes a first channel member 110 and a second channel member 120, which are pivotally connected at a single pivot point 130. In this embodiment, inserter 100 is configured to receive a disc nucleus implant 10 such as depicted in FIGS. 1-3. A first post extends radially inward from a sidewall of first channel member 110 and a second post extends radially inward from a sidewall of second channel member 120 in the loading region of the inserter (in a manner similar to the inserter embodiment of FIG. 7). These posts are sized and positioned to extend through respective apertures 24, 26 (see FIGS. 1-3) in implant 10. Thus, during transitioning of the inserter from the illustrated loading configuration to an implanting configuration, the posts extending radially within the first channel member and second channel member pivot away from each other, resulting in a straightening of the implant body within the passageway defined by first channel member 110 and second channel member 120.

In this example, first channel member 110 and second channel member 120 are pivotally connected at a single pivot point 130 at respective first end regions 112, 122. First channel member 110 and second channel member 120 further include respective second end regions 114, 124. Second end region 114 of first channel member 110 comprises the distal end region of inserter 100, while second end region 124 of second channel member 120 comprises the proximal end region of inserter 100. Second end region 114 of first channel member 110 is shown to have a conical-shaped outer surface 115 and the passageway within inserter 100 exits through an opening 116 in a side surface thereof. This opening is at other than at the apex of the conical-shaped outer surface in the second end region 114 of first channel member 110. In this embodiment, one or more slits 118 may be provided in the conical-shaped outer surface at the second end region 114 of first channel member 110. These slits 118 are positioned and of sufficient length to define flexible fingers that expand out when the nucleus replacement disc material is pushed through the passageway exit, thereby facilitating discharging of the nucleus replacement disc material. The apex of the conical-shaped outer surface is non-pointed, being blunt, or even rounded, to minimize the possibility of puncturing or penetrating the anterior annulus during dilation and insertion of the instrument, or during delivery of the nucleus material. Again, by way of example, a radius of a rounded apex at the distal end of the inserter is not less than 0.5 mm, and preferably not less than 1 mm.

In the illustrated embodiment, inserter 100 employs a single cover plate 140 pivoted 145 to first channel member 110 intermediate the first end region 112 and second end region 114 thereof. An elongate viewing window 119 is provided in first channel member 110 in order to provide a surgeon with feedback on the location of a nucleus replacement material within the inserter as the material is pushed through the passageway.

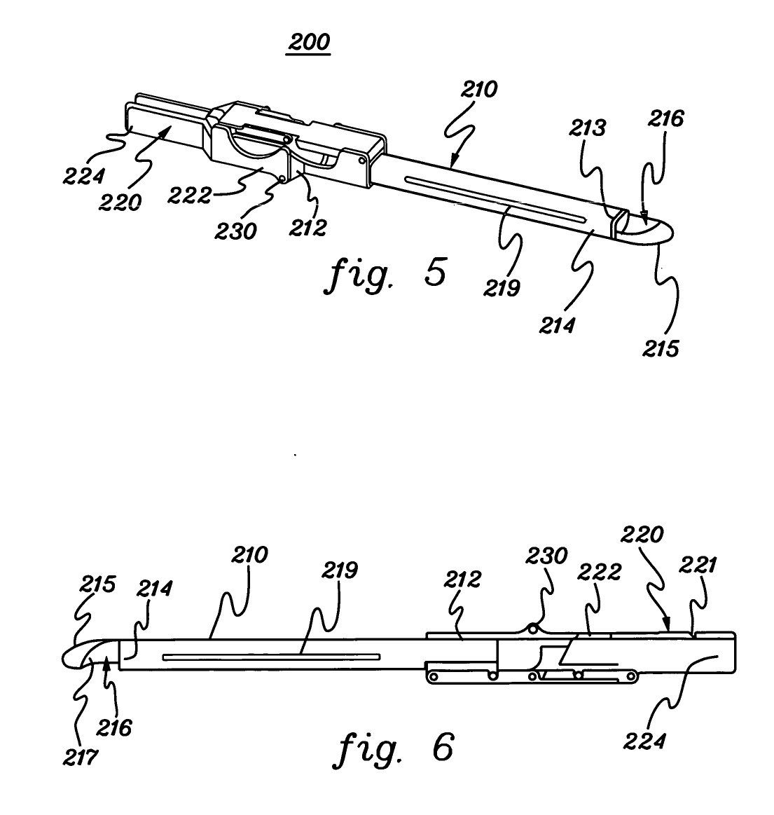

FIGS. 5-8 depict an alternate embodiment of an inserter, generally denoted 200, in accordance with certain aspects of the present invention. Referring first to FIGS. 5 & 6, this inserter 200 again includes a first channel member 210 and a second channel member 220, each having respective first (212, 222) and second (214, 224) end regions. Each channel member defines a passageway from its first end region to its second end region. The passageway within the first channel member is defined by at least one sidewall of the first channel member, while the passageway within the second channel member is defined by at least one sidewall of the second channel member. In the illustrated figures, the first channel member and the second channel member are pivotally connected at a single pivot point 230 at their respective first end regions, 212, 222. FIGS. 5 & 6 depict inserter 200 in an implanting configuration with the first channel member and the second channel member pivoted to define an angle of approximately of 180 degrees. The inserter assumes a loading configuration (see FIG. 7) when the first channel member and the second channel member are pivoted to define an angle of less than 180 degrees. When in the implanting configuration, the inserter passageway extends from second end region 224 of the second channel member through to a passageway exit 216 in a side surface at the second end of region 214 of the first channel member. In this configuration, second end region 224 of second channel member 222 is a proximal end region of inserter 200, while second end region 214 of first channel member 210 is a distal end region of inserter 200.

As shown, the distal end region of inserter 200 includes a conical-shaped outer surface 215 and an opening 216 in a side surface thereof through which the inserter passageway exits. In this embodiment, the apex of the conical-shaped exterior surface 215 is a solid surface, and a curved inner surface 217 is provided at opening 216 in the region of the conical-shaped outer surface to facilitate exiting of the nucleus replacement material from the passageway when the distal end region of the inserter is disposed in an intervertebral disc nucleus space. This inserter embodiment thus has a solid tip or apex in the distal end region of the inserter which has a wall thickness between the conical-shaped outer surface 215 and the curved inner surface 217 that is greater than a sidewall thickness of the inserter intermediate the proximal and distal end regions thereof. In this embodiment, the conical-shaped outer surface 215 at the distal end region of the inserter is sized to function as a dilator for dilating an opening in a disc annulus surrounding the intervertebral disc nucleus space as the distal end region of the inserter passes therethrough.

As with the embodiment of FIG. 4, the apex of the conical-shaped outer surface is non-pointed, for example, being blunt or rounded, to minimize the possibility of puncturing or penetrating the anterior annulus during dilation and insertion of the instrument, or during delivery of the nucleus material. A radius of a rounded apex of not less than 0.5 mm, or alternatively, not less than 1 mm, is preferred.

An elongate viewing window 219 is provided in first channel member 210 of inserter 200 to allow a surgeon visibility to the position of the nucleus replacement material within the inserter as the material is moving through the passageway towards passageway exit 216. Further, one or more visible depth markings 213 could be provided along the outer body of the inserter for monitoring penetration depth of the distal end region into the interbody space.

One embodiment of a cover locking mechanism for inserter 200 is illustrated in the partially enlarged, isometric view of inserter 200 shown in FIG. 7. In this illustration, the inserter is shown pivoting from a loading configuration, wherein first channel member 210 and second channel member 220 are pivoted to define an angle of less than 180 degrees, towards an implanting configuration wherein first channel member 210 and second channel member 220 are pivoted to an angle of approximately 180 degrees. Also, note that a first post 310 extending radially inward from the sidewall of first channel member 210, and a second post 320 extending radially inward from the second channel member 220 are illustrated in this figure. Posts 310 & 320 are employed in a manner as described above in connection with the inserter embodiment of FIG. 4. In particular, each post receives a respective aperture in a disc nucleus implant 10, such as depicted in FIGS. 1-3.

The cover locking mechanism includes a first cover plate 410 and a second cover plate 420. The first cover plate has a proximal end 412 and a distal end 414, while the second cover plate 420 has a proximal end 422 and a distal end 424. The distal end 414 of first cover plate 410 is pivotally connected 415 to first channel member 210 near the first end region thereof, while second cover plate 420 is pivotally connected 423 at the proximal end 422 thereof to second channel member 220 near the first end region of second channel member 220. Further, distal end 424 of second cover plate 420 is pivotally connected 425 to first cover plate 410 intermediate the proximal 412 and distal 414 ends of first cover plate 410. First cover plate 410 is configured with a latching tab 430 at proximal end 412 thereof. Further, second cover plate 420 is configured with a tab receiving landing 440 adjacent to proximal end 422 thereof.

The latching tab 430 and tab receiving landing 440 are sized, positioned and configured to mate and lock as the first cover plate and second cover plate are brought together simultaneous with the inserter transitioning to the implanting configuration from the loading configuration. This is accomplished, in one embodiment, by employing a rake angle lock between latching tab 430 and landing 440. For example, an edge surface 431 of latching tab 430 is configured with a rake angle, and an opposing wall surface 441 of second cover plate 420 defining a portion of tab receiving landing 440 is configured with a corresponding rake angle. Thus, as the inserter transitions to the implanting configuration, the first cover plate engages the second cover plate with the latching tab 430 mating to the landing 440, and the rake angled edge surface 431 entering opposing relation to the rake angled wall surface 441 of the second cover plate.

To facilitate unlocking of the cover locking mechanism, an opening in at least one of first cover plate 410 and second cover plate 420 accommodating pivot pin 425 is configured fractionally oversized to allow for movement between the first cover plate and the second cover plate when in the locked position and to thereby facilitate unlocking of the first and second cover plates. This fractional oversizing is sufficient to allow the inserter to slightly hyper-extend past the 180 degree implanting configuration to facilitate disengaging of the rake angle lock between surface 431 and surface 441 of the first and second cover plates, respectively.

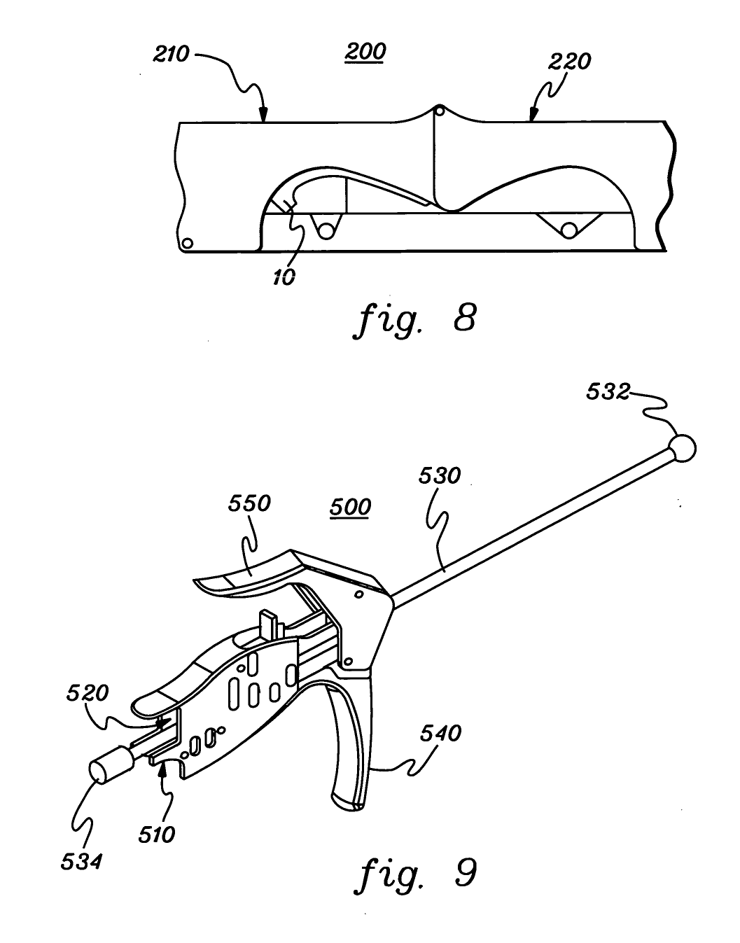

FIG. 8 is a partial depiction of inserter 200 of FIGS. 5-7 in the implanting configuration with the first and second channel members 210, 220 pivoted to align at approximately 180 degrees, and with a nucleus replacement disc 10 (such as illustrated in FIGS. 1-3) disposed therein. As illustrated, once in the implanting configuration, the nucleus replacement disc is substantially straightened, even more so than as depicted in FIG. 3. The disc is moved down the passageway of the inserter and out the passageway exit at the distal end region thereof through the side surface opening by employing a pusher rod and handle assembly such as described below.

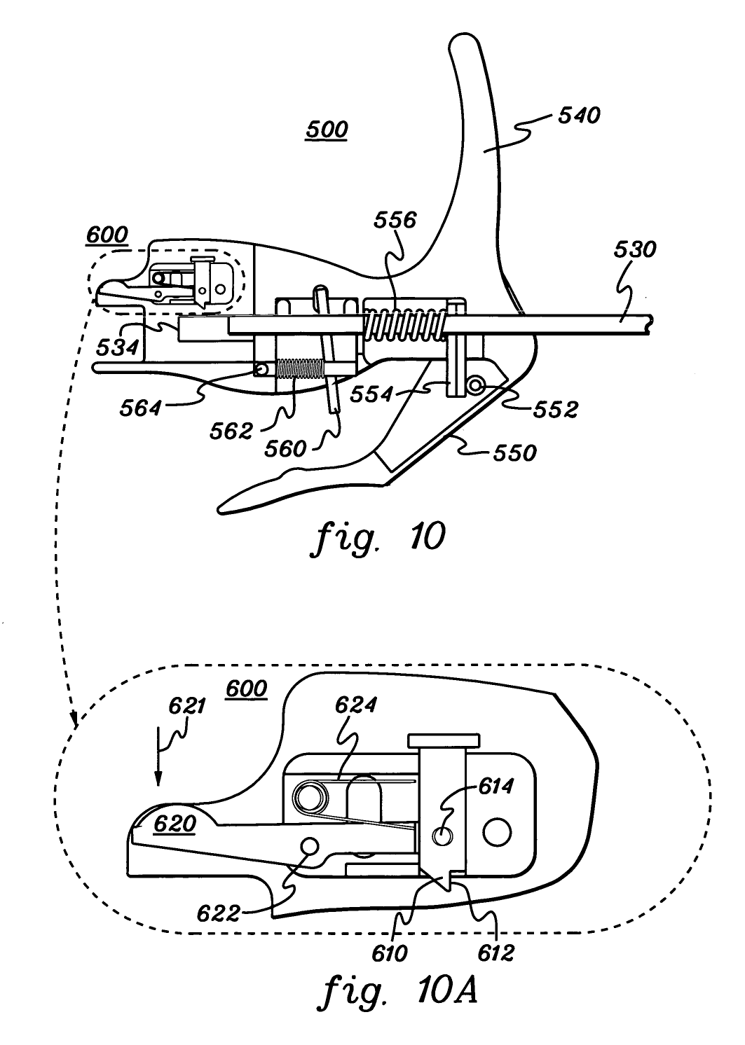

One embodiment of a handle assembly configured for releasable engagement with an inserter, such as inserter 200 of FIGS. 5-8, is depicted in FIGS. 9-10A. Referring first to FIG. 9, one embodiment of a ratchet handle assembly, generally denoted 500, is illustrated. An end 510 of handle assembly 500 is configured to releasably engage the proximal end region of the inserter, and includes a channel 520 through which pusher rod 530 reciprocates. When the inserter is releasably engaged by handle assembly 500, channel 520 aligns with the passageway through the inserter so that pusher rod 530 can extend into the inserter passageway. In this embodiment, pusher rod 530 includes a knob 532 on the proximal end thereof, and a pusher rod tip 534 on its distal end. A handle 540 facilitates manipulation of the instrument by a surgeon, and a lever 550 is provided to advance pusher rod 530 and apply a pushing force to the nucleus replacement material disposed within the passageway of the inserter when the instrument is in use. Multiple side surface openings in handle assembly 500 are illustrated in FIG. 9. These side openings facilitate cleaning and sterilization of the assembly 500.

FIGS. 10 & 10A depict in greater detail one embodiment of handle assembly 500, in accordance with aspects of the present invention. As shown, lever 550 of ratchet handle assembly 500 contacts a roller 552 which pushes against one or more driver plates 554. As the trigger lever is squeezed, roller 552 rolls across driver plates 554, thereby pushing the plates forward, locking onto pusher rod 530 and forcing the pusher rod forward as well. In this example, the forward direction is right to left. A pusher rod spring 556 returns the driver plates to a non-actuated position when the trigger lever 550 is released. A release plate 560 also engages pusher rod 530 and prevents the rod from prematurely retracting from applying pushing force to the nucleus replacement material within the inserter passageway. Release plate 560 is biased by a release spring 562 as shown. A positioning pin 564 positions the release plate 560 and spring 562 as needed for proper operation.

As shown in this figure, pusher tip 534 at the distal end of pusher rod 530 has a center axis somewhat offset from the center axis of pusher rod 530. This asymmetrical disposition of the pusher tip 534 axis relative to the pusher rod 530 axis facilitates forcing of a nucleus replacement disc through the inserter passageway where the nucleus replacement disc has a non-uniform profile within the passageway. Offsetting of the center axis of pusher tip 534 may be employed to match an equivalent center line across different nucleus replacement materials to be implanted employing the instrument. As noted above, different inserters with different sized inserter passageways may be made available for different sized implants, while the ratcheting handle assembly may be designed to accommodate various ones or all of the different sized inserters.

FIG. 10A is an enlarged depiction of one embodiment of a spring-biased latching mechanism 600 for ratcheting handle assembly 500, in accordance with aspects of the present invention. Mechanism 600 includes a latch member 610 configured to engage an inserter, and in particular, a groove in the proximal end region of the inserter when the inserter is releasably engaged by the handle assembly. (FIG. 6 depicts one embodiment of the groove 221 in the proximal end of the inserter 200. As shown, the groove is also angled to accommodate the angled latch member 610 therein.)

Surface 612 is oriented substantially perpendicular to the inserter and comprises the latching surface between the handle assembly and the inserter. Latch member 610 is pivotally connected 614 to a latch release lever 620. Latch release lever 620 is pivotally connected 622 intermediate its ends to the handle assembly. A spring 624 biases the latch release lever 620 and latch member 610 downward to ensure good engagement of the latch member with the groove in the inserter. When the latch release lever is actuated downward (in direction 621), the distal end of the latch release lever is pivoted upwards, thereby retracting the latch member 610 from the groove in the proximal end region of the inserter, and allowing detachment of the handle assembly from the inserter.

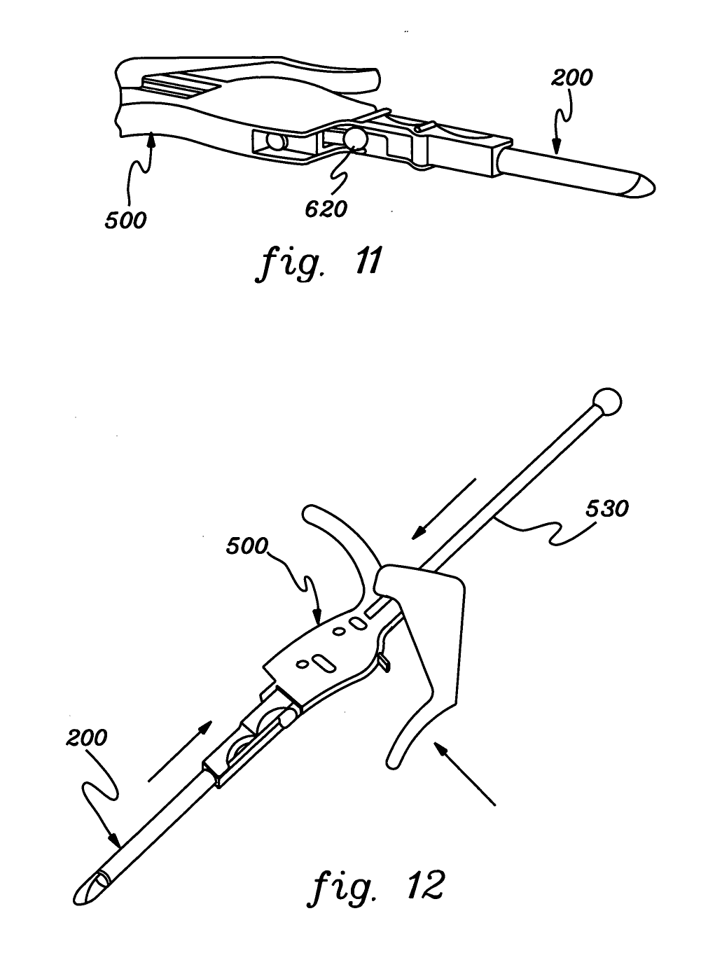

FIG. 11 depicts one partial embodiment of the implanting instrument with the ratcheting handle assembly releasably engaging the inserter, and illustrating one position for the latch release lever of the spring-biased latch mechanism.

FIGS. 12-14 illustrate one embodiment of the assembled instrument, generally denoted 700, which includes inserter 200 (see FIGS. 5-8) and ratcheting handle assembly 500 (see FIGS. 9-10A). Initially, a loaded inserter 200 is placed into operable engagement with ratcheting handle assembly 500 (see FIG. 12) and is held in position by the spring-biased latching mechanism thereof described above. When in operable engagement, pusher rod 530 is stepwise actuated to apply pushing force, via the pusher tip at the distal end thereof, to the nucleus replacement material 10 (for example, a nucleus replacement disc such as illustrated in FIGS. 1-3) positioned within the passageway of the inserter.

The material can be implanted from the instrument by providing an appropriate hole or opening in the annulus of a disc receiving the material for replacing or augmenting of the intervertebral disc nucleus. The hole or opening in the annulus is assumed to have an undilated size that may be smaller than the cross-sectional size of the material for replacing or augmenting the intervertebral disc nucleus. The hole has a dilated size that is larger than the cross-sectional size of the material, disposed within the inserter, for replacing or augmenting the intervertebral disc nucleus. This is achieved by the distal end region of the inserter dilating the hole in the disc annulus as the inserter passes into the hole. Introducing the distal end region of the inserter interbody includes positioning at least a portion of the passageway exit at the distal end region of the inserter within the intervertebral disc nucleus space as shown in FIG. 14. In one implementation, the distal end region of the inserter is introduced into the intervertebral disc nucleus space with the passageway exit at the distal end region fully within the disc space prior to delivery of the nucleus replacement material. By doing so, the proximal (i.e., trailing) end of the nucleus replacement material will be properly deposited within the disc space. One or more visible depth markings along the outer body of the inserter could be employed for monitoring penetration depth of the distal end region into the interbody space. Further, those skilled in the art will note that the passageway exit at the distal end region of the inserter should be facing the nuclear disc void created during disectomy/nucleotomy (i.e., facing medial) when employing a nucleus replacement material such as illustrated in FIGS. 1-3, wherein the implant folds or recoils as it enters the disc space.

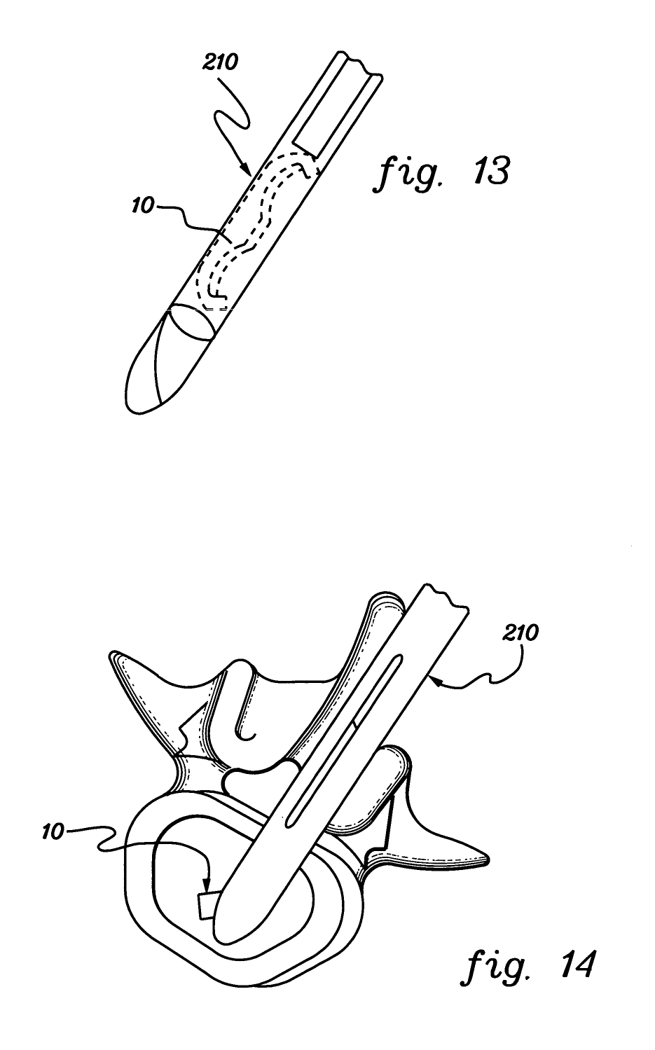

By actuating the ratchet lever, the pusher rod advances the nucleus replacement material down the passageway of the inserter (see FIG. 13) and out the passageway exit in the distal end region thereof, as shown in FIG. 14. If desired, the nucleus replacement material can be positioned down the passageway in the inserter prior to inserting of the distal end region of the inserter into the annular opening. Once inserted within the annular opening, the nucleus replacement material is extruded through the passageway exit in the side surface of the inserter.

### EXAMPLE

A medical patient may be treated to replace a damaged or degenerated lumbar intervertebral disc nucleus using the procedure described above.

A/P and M/L radiographs are obtained to determine the size and shape of the affected level. The largest implant that can be accommodated by patient anatomy without overdistraction is selected, choosing (for example) among implants having footprints of 19 mm×23 mm to 22 mm×27 mm, and a height of between 6 mm and 14 mm. It is important to select the tallest device that can be accommodated by the interbody space. Excessive annulus laxity may cause non-central seating of the implant. X-ray templates are used to determine whether a small or large device footprint should be used, as are AP and ML implant outlines to determine the appropriate height.

The patient is placed in a direct prone position on the operating table, bolstered appropriately to maintain lumbar lordosis. C-arm fluoroscopy is not absolutely necessary for the procedure, but is preferred if available. Intraoperative imaging is useful for evaluation of the nucleus cavity preparation, as well as for adjusting and confirming device orientation.

A 5 cm incision is made in the midline directly over the posterior spinous processes. The skin incision is sharply carried down through the subcutaneous tissues to the dorsal lumbar fascia. Great care is taken to preserve the midline ligamentous structures. A longitudinal incision is made in the dorsal lumbar fascia 5 mm lateral to the posterior spinous processes. The multifidus is subperiostally elevated off the posterior spinous processes and adjacent lamina. Great care is taken to protect and preserve the facet joint capsule and joint.

A high speed burr is used to create a small laminotomy window. The ligamentum flavum is sharply incised and removed. A Kerrison rongeur is used to enlarge the laminotomy site if necessary. The traversing nerve root is identified and gently retracted medially.

Epidural veins are coagulated using bipolar electrocaurtery. The posterior annulus is identified. A working portal through the annulus is created following insertion of the trephine device.

Preservation of the annulus fibrosis minimizes the risk of implant expulsion. A progressive dilation technique is employed to gain access to the nucleus pulposus. If properly dilated and protected, the viscoelastic annulus fibers should relax postoperatively, leaving only a small defect.

A starting hole is created in the annulus using a 3 mm trephine. The first dilator is then inserted, taking care not to damage the anterior margin of the annulus. Larger dilators are then provided over each shaft in sequence until the desired access is achieved.

A variety of tools are used to properly clear the nucleus cavity, including specialized pituitary rongeurs and curettes for reaching the contralateral margin of the nucleus pulposus. Ring curettes are used to scrape adhesions from the vertebral endplates if necessary. Care is taken to thoroughly prepare the cavity such that it is centralized, symmetrical, and large enough to accept the desired implant footprint. Care is also taken to avoid damaging the annulus fibrosis.

The endplate jack is inserted into the intervertebral space and is actuated until moderate distraction is achieved. Care is taken to avoid overdistraction. The position is maintained for approximately 60 seconds to allow the annulus fibers to relax, adjusting if necessary during the process. The height on the jack scale is identified and the corresponding implant is selected. When the desired implant falls between sizes, the smaller implant size is selected.

An instrument set containing numerous device inserter bodies, with internal geometry specific to corresponding implants, is used to insert the implant. All inserter bodies interface with a common ratchet assembly and pusher rod, as described above. The inserter body is chosen to correspond to the correct implant size and the implant is loaded into the instrument. The instrument is then straightened to its implanting configuration.

The inserter functions much like a caulking gun. The loaded inserter body is assembled with the ratchet handle, and the pusher rod is positioned into the ratchet handle until it touches the nucleus replacement device. The ratchet handle assembly is then actuated to advance the implant to a position just before the shorter foot of the inserter pivot. This minimizes the time and travel required for insertion once the instrument is installed at the operative site. If an implant is accidentally advanced to the point where the shorter foot begins to open, the implant is extruded out of the device and the inserter is reloaded.

The inserter tip is placed in the annular opening prior to extruding the nucleus replacement device beyond the pivot point of the shorter foot. The inserter is then positioned such that the stationary portion is lateral and the pivoting shorter foot is medial. This allows the implant to curl into the prepared space as it is extruded out of the inserter. As the nucleus replacement device fills the nucleus cavity, it will tend to push the inserter out of the disc space. Moderate axial force is applied during the final stage of extrusion to counter this effect. If the trailing edge of the nucleus replacement device protrudes slightly from the annulus following insertion, it can be easily pushed into closed position.

Further, using fluoroscopic control, the final position of the nucleus replacement material can be adjusted using a tamp. Positioning can be verified by inspection of the radiographic markers embedded in the disc. As noted above, the anterior marker is slightly larger than the two posterior markers. If ideally placed, three collinear markers are visible in the frontal plane, with the central marker being larger than the outer two. In the sagittal plane, a larger anterior marker and two closely positioned posterior marker are visible. This ideal placement may not be necessary because the implant will float and rotate slightly as it finds a natural center in the nucleus space.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the following claims.

What is claimed is:

1. An instrument for implanting nucleus replacement material into an intervertebral disc nucleus space, the instrument comprising:
an inserter including a passageway effective for passing nucleus replacement material therethrough, the inserter having a proximal end region and a distal end region; and
wherein the distal end region of the inserter comprises a conical-shaped outer surface, and wherein the passageway exits through a side surface of the inserter in the distal end region thereof at other than the apex of the conical-shaped outer surface, and wherein nucleus replacement material passing through the inserter exits into an intervertebral disc nucleus space when the passageway exit in the side surface of the inserter in the distal end region is at least partially disposed in the intervertebral disc nucleus space.

2. The instrument of claim 1, wherein the apex of the conical-shaped outer surface at the distal end region of the inserter comprises a blunt, solid apex surface, and wherein the blunt, solid apex surface and the conical-shaped outer surface are sized to function as a dilator for dilating an opening in a disc annulus surrounding the intervertebral disc nucleus space as the distal end region of the inserter passes therethrough.

3. The instrument of claim 2, wherein the blunt, solid apex surface comprises a rounded apex surface having a radius of curvature greater than 0.5 mm.

4. The instrument of claim 2, wherein the distal end region of the inserter further comprises a curved inner surface in the region of the conical-shaped outer surface, and wherein a thickness of the inserter at the distal end region between the conical-shaped outer surface and the curved inner surface is greater than a sidewall thickness of the inserter intermediate the proximal end region and distal end region thereof.

5. The instrument of claim 4, wherein the curved inner surface of the inserter at the distal end region is configured to facilitate exiting of the nucleus replacement material from the passageway into the intervertebral disc nucleus space when the distal end region of the inserter is disposed in the intervertebral disc nucleus space.

6. The instrument of claim 1, wherein the apex and the conical-shaped outer surface are sized to function as a dilator for dilating an opening of a disc annulus surrounding the intervertebral disc nucleus space as the distal end region of the inserter passes therethrough, the apex and the conical-shaped outer surface at the distal end region further including at least one slit therein to facilitate passage of the nucleus replacement material through the passageway exit in the side surface of the inserter at the distal end region, and thereby facilitate placement of the nucleus replacement material within the intervertebral disc nucleus space.

7. The instrument of claim 1, wherein the inserter further comprises an elongate viewing window disposed in a sidewall thereof to allow viewing of the nucleus replacement material within the passageway, the elongate viewing window being disposed intermediate the proximal and distal end regions of the inserter.

8. The instrument of claim 1, wherein the inserter further comprises a first channel member and a second channel member pivotally connected together at respective first ends thereof, wherein the inserter assumes a loading configuration when the first channel member and the second channel member are pivoted to define an angle of less than 180 degrees, and assumes an implanting configuration when the first channel member and the second channel member are pivoted to define an angle of approximately 180 degrees, and wherein the first and second channel members define the passageway when in the implanting configuration.

9. The instrument of claim 8, wherein the distal end region of the inserter comprises the second end of the first channel member, and the proximal end region of the inserter comprises the second end of the second channel member, and wherein the first channel member and the second channel member pivotally connect at their first ends about a single pivot point.

10. The instrument of claim 9, further comprising a first post extending radially inward from at least one sidewall of the first channel member and a second post extending radially inward from at least one sidewall of the second channel member, wherein the first post and the second post are sized and positioned to facilitate loading of the nucleus replacement material within the inserter when in the loading configuration.

11. The instrument of claim 10, wherein the nucleus replacement material comprises a prosthetic nucleus replacement disc including a pair of arms folded to form an inner fold when the prosthetic nucleus replacement disc is in a relaxed configuration, and wherein at least two apertures are provided in the nucleus replacement disc, each sized and positioned to receive a respective one of the first post and second post when the nucleus replacement disc is loaded into the inserter, wherein when the inserter assumes the implanting configuration with the prosthetic nucleus replacement disc disposed therein, the prosthetic nucleus replacement disc straightens to a cross-sectional size that is less than a cross-sectional size of the prosthetic nucleus replacement disc in the relaxed configuration, the straightened cross-sectional size allowing for movement of the prosthetic nucleus replacement disc through the passageway.

12. The instrument of claim 9, further comprising a cover locking mechanism comprising a first cover plate and a second cover plate, the first cover plate comprising a proximal end and a distal end, the distal end of the first cover plate being pivotally connected to the first channel member near the first end thereof, and wherein the second cover plate comprises a proximal end and a distal end, the proximal end of the second cover plate being pivotally connected to the second channel member near the first end thereof, and the distal end of the second cover plate being pivotally connected to the first channel plate intermediate the proximal and distal ends of the first cover plate, and wherein the first and second cover plates are configured with a latching mechanism at the proximal ends thereof, wherein the latching mechanism automatically latches the first cover plate to the second cover plate when the inserter assumes the implanting configuration.

13. The instrument of claim 12, wherein the latching mechanism comprises a latching tab disposed at the proximal end of the first cover plate and a tab receiving landing in the second cover plate, the latching tab being sized and configured to mate and lock to the tab receiving landing in the second cover plate as the inserter assumes the implanting configuration.

14. The instrument of claim 13, wherein the latching tab comprises an edge surface with a rake angle, and wherein the tab receiving landing is at least partially defined by a wall surface of the second cover plate having a corresponding rake angle to the rake angle of the latching tab edge surface, the surfaces with the corresponding rake angles forming a rake angle lock as the latching tab engages the tab receiving landing.

15. The instrument of claim 14, wherein the distal end of the second cover plate pivotally connects to the first cover plate via a pivot pin, and wherein the pivot pin passes through an opening in at least one of the first cover plate and the second cover plate, the opening being fractionally oversized to allow for movement between the first cover plate and second cover plate when in a locked position to facilitate unlocking of the first cover plate and second cover plate.

16. The instrument of claim 8, further comprising a handle assembly releasably engaging a second end of the second channel member at the proximal end region of the inserter, the handle assembly comprising a pusher rod aligned with the passageway of the inserter in the implanting configuration, the pusher rod including a pusher rod tip at a distal end thereof, the pusher rod tip engaging the nucleus replacement material when in operation to move the nucleus replacement material through the passageway, out the passageway exit, and into the intervertebral disc nucleus space.

17. The instrument of claim 16, further comprising a spring-biased latching mechanism for latching the handle assembly to the proximal end region of the inserter, the spring-biased latching mechanism comprising a latch member coupled to the handle assembly and engaging a groove in the proximal end region of the inserter to releasably attach the inserter to the handle assembly, and wherein the latch member is pivotally coupled to a latch release lever, the latch release lever being spring-biased to ensure engagement of the latch member with the groove in the inserter, wherein when the latch release lever is actuated, the latch release lever pivots to retract the latch member from the groove in the proximal end region of the inserter, thereby allowing detachment of the handle assembly from the inserter.

18. An instrument for implanting a nucleus replacement disc, the instrument comprising:
a first channel member having a first end region and a second end region, the first channel member defining a first passageway from the first end region to the second end region, the first passageway being defined by at least one sidewall;
a first post extending radially inward from the at least one sidewall of the first channel member, the first post being located at the first end region of the first channel member;
a second channel member having a first end region and a second end region, the second channel member defining a second passageway from the first end region to the second end region thereof, the second passageway being defined by at least one sidewall of the second channel member;
a second post extending radially inward from the at least one sidewall of the second channel member, the second post being located at the first end region of the second channel member;
wherein the first channel member and the second channel member are pivotally connected at their respective first end regions, and wherein the instrument assumes a loading configuration when the first channel member and the second channel member are pivoted to define an angle of less than 180 degrees, and assumes an implanting configuration when the first channel member and the second channel member are pivoted to define an angle of approximately 180 degrees, wherein in the implanting configuration the first passageway and the second passageway align to form a single aligned passageway; and
wherein the second end region of the first channel member comprises a conical-shaped outer surface and the aligned passageway exits through a side surface of the first channel member at the second end region thereof at other than the apex of the conical-shaped outer surface, wherein a nucleus replacement disc passing through the aligned passageway exits into an intervertebral disc nucleus space when the passageway exit at the second end region of the first channel member is at least partially disposed in the intervertebral disc nucleus space.

19. The instrument of claim 18, wherein the apex of the conical-shaped outer surface at the second end region of the first channel member comprises a blunt, solid apex surface, the blunt, solid apex surface and the conical-shaped outer surface being sized to function as a dilator for dilating an opening in a disc annulus surrounding the intervertebral disc nucleus space as the second end region of the first channel member passes therethrough.

20. The instrument of claim 19, wherein the blunt, solid apex surface comprises a rounded apex surface having a radius of curvature greater than 0.5 mm.

21. The instrument of claim 19, wherein the first channel member further comprises a curved inner surface in the second end region having the conical-shaped outer surface, and wherein a thickness of the inserter between the conical-shaped outer surface and the curved inner surface is greater than a sidewall thickness of the inserter at the first channel member intermediate the first and second ends thereof.

22. The instrument of claim 21, wherein the curved inner surface of the first channel member is configured to facilitate exiting of the nucleus replacement disc from the aligned passageway into the intervertebral disc nucleus space when the second end region of the first channel member is disposed in the intervertebral disc space.

23. The instrument of claim 18, wherein the apex and the conical-shaped outer surface are sized to function as a dilator for dilating an opening in a disc annulus surrounding the intervertebral disc nucleus space as the second end region of the first channel member passes therethrough, the apex and the conical-shaped outer surface at the second end region of the first channel member further including at least one slit therein to facilitate passage of the nucleus replacement disc through the passageway exit in the side surface of the first channel member at the second end region thereof, thereby facilitating placement of the nucleus replacement disc within the intervertebral disc nucleus space.

24. The instrument of claim 18, wherein the first channel member further comprises an elongate viewing window disposed in a sidewall thereof to allow viewing of the nucleus replacement disc within the aligned passageway, the elongate viewing window being disposed intermediate the first and second end regions of the first channel member.

25. The instrument of claim 18, wherein the nucleus replacement disc comprises a prosthetic nucleus replacement disc including a pair of arms folded to form an inner fold when the nucleus replacement disc is in a relaxed configuration, and wherein at least two apertures are provided in the nucleus replacement disc, each sized and positioned to receive a respective one of the first post and second post when the prosthetic nucleus replacement disc is loaded into the instrument, wherein when the instrument assumes an implanting configuration with the prosthetic nucleus replacement disc positioned therein, the prosthetic nucleus replacement disc straightens to a cross-sectional size that is less than a cross-sectional size of the prosthetic nucleus replacement disc in the relaxed configuration, the straightened cross-sectional size allowing for movement of the prosthetic nucleus replacement disc through the aligned passageway.

26. The instrument of claim 18, wherein the first channel member and the second channel member pivotally connect at their first ends about a single pivot point.

27. The instrument of claim 18, further comprising a cover locking mechanism comprising a first cover plate and a second cover plate, the first cover plate comprising a proximal end and a distal end, the distal end of the first cover plate being pivotally connected to the first channel member at the first end region thereof, and wherein the second cover plate comprises a proximal end and a distal end, the proximal end of the second cover plate being pivotally connected to the second channel member at the first end region thereof, and the distal end of the second cover plate being pivotally connected to the first channel plate intermediate the proximal and distal ends of the first cover plate, and wherein the first and second cover plates are configured with a latching mechanism near the proximal ends thereof, wherein the latching mechanism automatically latches the first cover plate to the second cover plate as the inserter assumes the implanting configuration.

28. The instrument of claim 27, wherein the latching mechanism comprises a latching tab disposed at the proximal end of the first cover plate and a tab receiving landing near the proximal end of the second cover plate, the latching tab being sized and configured to mate and lock to the tab receiving landing in the second cover plate as the inserter assumes the implanting configuration.

29. The instrument of claim 28, wherein the latching tab comprises an edge surface with a rake angle, and wherein the tab receiving landing is at least partially defined by a wall surface of the second cover plate having a corresponding rake angle to the rake angle of the latching tab edge surface, the surfaces with the corresponding rake angles forming a rake angle lock as the latching tab engages the tab receiving landing.

30. The instrument of claim 29, wherein the distal end of the second cover plate pivotally connects to the first cover plate via a pivot pin, and wherein the pivot pin passes through an opening in at least one of the first cover plate and the second cover plate, the opening being fractionally oversized to allow for movement between the first cover plate and second cover plate when in a locked position to facilitate unlocking of the first cover plate and second cover plate.

31. The instrument of claim 18, further comprising a handle assembly releasably engaging the second end region of the second channel member, the handle assembly comprising a pusher rod reciprocal within the aligned passageway of the instrument when in the implanting configuration, the pusher rod including a pusher rod tip at a distal end thereof, the pusher rod tip engaging the nucleus replacement disc when in operation to move the nucleus replacement disc through the aligned passageway, out the passageway exit, and into the intervertebral disc nucleus space.

32. The instrument of claim 31, further comprising a spring-biased lathing mechanism for latching the handle assembly to the second end region of the second channel member, the spring-biased latching mechanism comprising a latch member coupled to the handle assembly and engaging a groove in the second end region of the second channel member to releasably attach the second channel member to the handle assembly, and wherein the latch member is pivotally coupled to a latch release lever, the latch release lever being spring-biased to ensure engagement of the latch member with the groove in the second end region of the second channel member, wherein when the latch release lever is actuated, the latch release lever pivots to retract the latch member from the groove in the second end region of the second channel member, thereby allowing detachment of the second channel member from the handle assembly.

33. An instrument for implanting nucleus replacement material, the instrument comprising:
an inserter defining a passageway effective for passing nucleus replacement material therethrough, the inserter having a proximal end region and a distal end region, and comprising a first channel member and a second channel member pivotally connected together at respective first ends thereof, wherein the inserter assumes a loading configuration when the first channel member and the second channel member are pivoted to define an angle of less than 180 degrees, and assumes an implanting configuration when the first channel member and the second channel member are pivoted to define an angle of approximately 180 degrees, and wherein the first and second channel members define the passageway when the inserter is in the implanting configuration; and
a cover locking mechanism comprising a first cover plate and a second cover plate, the first cover plate comprising a proximal end and a distal end, the distal end of the first cover plate being pivotally connected to the first channel member near the first end thereof, and wherein the second cover plate comprises a proximal end and a distal end, the proximal end of the second cover plate being pivotally connected to the second channel member near the first end thereof and the distal end of the second cover plate being pivotally connected to the first cover plate intermediate the proximal and distal ends of the first cover plate, and wherein the first cover plate is configured with a latching tab at the proximal end thereof, and the second cover plate is configured with a tab receiving landing, the latching tab and the tab receiving landing being sized and configured to mate and lock the first cover plate and second cover plate together as the inserter assumes the implanting configuration.

34. The instrument of claim 33, wherein the latching tab comprises an edge surface with a rake angle, and wherein the tab receiving landing is at least partially defined by a wall surface of the second cover plate having a corresponding rake angle to the rake angle of the latching tab edge surface, the surfaces with the corresponding rake angles forming a rake angle lock as the latching tab engages the tab receiving landing.

35. The instrument of claim 34, wherein the distal end of the second cover plate pivotally connects to the first cover plate via a pivot pin, and wherein the pivot pin plate, the opening being fractionally oversized to allow for movement between the first cover plate and the second cover plate when in a locked position to facilitate unlocking of the first cover plate and the second cover plate.

36. The instrument of claim 33, wherein the first channel member and the second channel member pivotally connect at their first ends about a single pivot point, and wherein the instrument further comprises a first post extending radially inward from at least one sidewall of the first channel member and a second post extending radially inward from at least one sidewall of the second channel member, wherein the first post and the second post are sized and positioned to facilitate loading of the nucleus replacement material when the instrument is in the loading configuration.

37. The instrument of claim 36, wherein the disc nucleus replacement material comprises a prosthetic nucleus replacement disc including a pair of arms folded to form an inner fold when the prosthetic nucleus replacement disc is in a relaxed configuration, and wherein at least two apertures are provided in the prosthetic nucleus replacement disc, each sized and positioned to receive a respective one of the first post and second post when the prosthetic nucleus replacement disc is loaded into the instrument, wherein when the instrument assumes an implanting configuration with the prosthetic nucleus replacement disc disposed therein, the prosthetic nucleus replacement disc straightens to a cross-sectional size that is less than a cross-sectional size of the prosthetic nucleus replacement disc in the relaxed configuration, the straightened cross-sectional size allowing for movement of the prosthetic nucleus replacement disc through the passageway.

38. The instrument of claim 33, further comprising a handle assembly releasably engaging a second end of the second channel member at the proximal end region of the inserter, the handle assembly comprising a pusher rod aligned to extend into the passageway when the inserter assumes the implanting configuration, the pusher rod including a pusher rod tip at a distal end thereof, the pusher rod tip engaging the nucleus replacement material when in operation to move the nucleus replacement material through the passageway, out a passageway exit in the inserter at the distal end region thereof, and into an intervertebral disc nucleus space when the passageway exit at the distal end region of the inserter is disposed in the intervertebral disc nucleus space.

39. The instrument of claim 38, further comprising a spring-biased latching mechanism for latching the handle assembly to the proximal end region of the inserter, the spring-biased latching mechanism comprising a latch member coupled to the handle assembly and engaging a groove in the proximal end region of the inserter to releasably attach the inserter to the handle assembly, and wherein the latch member is pivotally coupled to a latch release lever, the latch release lever being spring-biased to ensure engagement of the latch member with the groove in the inserter, wherein when the latch release lever is actuated, the latch release lever pivots to retract the latch member from the groove in the proximal end region of the inserter, thereby allowing detachment of the handle assembly from the inserter.

40. The instrument of claim 38, wherein the distal end region of the inserter comprises a conical-shaped outer surface, and wherein the passageway exits through a side surface of the inserter in the distal end region thereof at other than the apex of the conical-shaped outer surface.

41. A method of implanting material in an intervertebral disc nucleus space, the method comprising:
providing a disc nucleus implant instrument including:
an inserter including a passageway effective for passing a material for replacing or augmenting an intervertebral disc nucleus, the inserter having a proximal end region and a distal end region; and
wherein the distal end region of the inserter comprises a conical-shaped outer surface, and wherein the passageway exits through a side surface of the inserter in the distal end region thereof at other than the apex of the conical-shaped outer surface, the distal end region of the inserter with the conical-shaped outer surface being sized to function as a dilator;
providing a material suitable for replacing or augmenting an intervertebral disc nucleus in the passageway of the inserter;
providing a hole in the annulus of a disc receiving the material for replacing or augmenting the intervertebral disc nucleus, the hole having an undilated size that is smaller than a cross-sectional size of the material for replacing or augmenting the intervertebral disc nucleus, the hole having a dilated size that is larger than the cross-sectional size of the material for replacing or augmenting the intervertebral disc nucleus;
introducing the dilating distal end region of the inserter into the hole in the disc annulus to dilate the hole in the disc annulus, the introducing comprising positioning the passageway exit of the distal end region of the inserter at least partially within the intervertebral disc nucleus space;
passing the material for replacing or augmenting the intervertebral disc nucleus through the passageway exit in the side surface of the inserter in the distal end region; and
withdrawing the inserter, and allowing the hole in the disc annulus to return to a smaller size than its dilated size.

42. The method of claim 41, wherein providing the disc nucleus implant instrument further comprises providing the inserter with a curved inner surface at the dilating distal end region, and wherein a thickness of the inserter at the apex of the dilating distal end region between the conical-shaped outer surface and the curved inner surface is greater than a sidewall thickness of the inserter intermediate the proximal end region and distal end region thereof.

43. The method of claim 42, wherein providing the disc nucleus implant instrument further comprises providing the curved inner surface at the dilating distal end region of the inserter in a configuration to facilitate exiting of the material from the passageway into the intervertebral disc nucleus space.

44. The method of claim 41, wherein providing the disc nucleus implant instrument providing the distal end region of the inserter with a blunt, sold apex surface, and wherein a radius of curvature of the blunt, solid apex surface is greater than 0.5 mm.

45. The method of claim 41, wherein providing the disc nucleus implant instrument further includes providing the inserter with an elongate viewing window disposed in a sidewall thereof to allow viewing of the material for replacing or augmenting the intervertebral disc nucleus when disposed within the passageway, the elongate viewing window being disposed intermediate the proximal and distal end regions of the inserter, and wherein providing the material suitable for replacing or augmenting the intervertebral disc nucleus in the passageway of the inserter further comprises positioning the material within the inserter adjacent to the passageway exit in the distal end region thereof.

46. The method of claim 41, wherein providing the disc nucleus implant instrument further comprises providing a first channel member and a second channel member pivotally connected together at respective first ends thereof to define the inserter, wherein the inserter assumes a loading configuration when the first channel member and the second channel member are pivoted to define an angle of less than 180 degrees, and assumes an implanting configuration when the first channel member and the second channel member are pivoted to define an angle of approximately 180 degrees, wherein the first and second channel members define the passageway of the inserter when in the implanting configuration, and wherein providing the material for replacing or augmenting the intervertebral disc nucleus further comprises loading the material for replacing or augmenting the intervertebral disc nucleus into the passageway with the inserter in the loading configuration, and thereafter, transitioning the inserter from the loading configuration to the implanting configuration.

47. The method of claim 46, wherein providing the disc nucleus implant instrument further comprises providing a first post extending radially inward from at least one sidewall of the first channel member and a second post extending radially inward from at least one sidewall of the second channel member, wherein the first post and the second post are sized and positioned to facilitate loading of the material for replacing or augmenting the intervertebral disc nucleus, and wherein the material for replacing or augmenting the intervertebral disc nucleus comprises a prosthetic nucleus replacement disc including a pair of arms folded to form an inner fold when the prosthetic nucleus replacement disc is in a relaxed configuration, and wherein at least two apertures are provided in the prosthetic nucleus replacement disc, each sized and positioned to receive a respective one of the first post and second post when the prosthetic nucleus replacement disc is loaded into the inserter, and wherein when the inserter assumes the implanting configuration with the prosthetic nucleus replacement disc disposed therein, the prosthetic nucleus replacement disc straightens to a cross-sectional size that is less than a cross-sectional size of the prosthetic nucleus replacement disc in the relaxed configuration, the straightened cross-sectional size allowing for movement of the prosthetic nucleus replacement disc through the passageway.

48. The method of claim 46, wherein providing the disc nucleus implant instrument further comprises providing a cover locking mechanism comprising a first cover plate and a second cover plate, the first cover plate comprising a proximal end and a distal end, the distal end of the first cover plate being pivotally connected to the first channel member near the first end thereof, and wherein the second cover plate comprises a proximal end and a distal end, the proximal end of the second cover plate being pivotally connected to the second channel member near the first end thereof, and the distal end of the second cover plate being pivotally connected to the first channel member intermediate the proximal and distal ends of the first cover plate, and wherein the first and second cover plates are configured with a latching mechanism adjacent to the proximal ends thereof, the latching mechanism comprising a latching tab disposed at the proximal end of the first cover plate and a tab receiving landing in the second cover plate, and wherein the method further comprises transitioning the inserter from the loading configuration to the implanting configuration, the transitioning comprising pivoting the first channel member and the second channel member to define an angle of approximately 180 degrees, and simultaneously therewith, pivoting the first cover plate towards the second cover plate so that the latching tab mates and locks with the tab receiving landing of the second cover plate.

49. The method of claim 41, wherein providing the disc nucleus implant instrument further comprises providing the instrument with a handle assembly releasably engaging the proximal end region of the inserter, the handle assembly comprising a pusher rod aligned with the passageway of the inserter when in the implanting configuration, and wherein the passing of the material for replacing or augmenting the intervertebral disc nucleus through the passageway exit in the side surface of the inserter in the distal end region thereof further comprises employing the pusher rod of the handle assembly to force the material from the passageway of the inserter through the passageway exit and into the intervertebral disc nucleus space.

50. The method of claim 41, wherein introducing the dilating distal end region of the inserter into the hole further comprises positioning the passageway exit of the distal end region of the inserter at least partially within the intervertebral disc nucleus space and facing medial.

51. A method of implanting material in an intervertebral disc nucleus space, the method comprising:
providing a disc nucleus implant instrument comprising:
an inserter including a passageway effective for passing material for replacing or augmenting an intervertebral disc nucleus, the inserter having a proximal end region and a distal end region, and comprising a first channel member and a second channel member pivotally connected together at respective first ends thereof, wherein the inserter assumes a loading configuration when the first channel member and the second channel member are pivoted to define an angle of less than 180 degrees, and assumes an implanting configuration when the first channel member and the second channel member are pivoted to define an angle of approximately 180 degrees, and wherein the first and second channel members define the passageway when the inserter is in the implanting configuration; and
a cover locking mechanism comprising a first cover plate and a second cover plate, the first cover plate comprising a proximal end and a distal end, the distal end of the first cover plate being pivotally connected to the first channel member near the first end thereof, and wherein the second cover plate comprises a proximal end and a distal end, the proximal end of the second cover plate being pivotally connected to the second channel member near the first end thereof and the distal end of the second cover plate being pivotally connected to the first cover plate intermediate the proximal and distal ends of the first cover plate, and wherein the first cover plate is configured with a latching tab at the proximal end thereof, and the second cover plate is configured with a tab receiving landing, the latching tab and the tab receiving landing being sized and configured to mate and lock the first cover plate and the second cover plate together as the inserter assumes the implanting configuration;
providing a material suitable for replacing or augmenting an intervertebral disc nucleus in the passageway of the inserter while the inserter is in the loading configuration;
transitioning the inserter from the loading configuration to the implanting configuration, the transitioning comprising pivoting the first channel member and the second channel member to define the angle of approximately 180 degrees, and simultaneous therewith, pivoting the first cover plate towards the second cover plate so that the latching tab of the first cover plate mates and locks with the tab receiving landing of the second cover plate;
providing a hole in the annulus of a disc receiving the material for replacing or augmenting an intervertebral disc nucleus;
introducing the distal end region of the inserter into the hole in the disc annulus, the introducing comprising positioning the distal end region of the inserter within the intervertebral disc nucleus space;
passing the material for replacing or augmenting the intervertebral disc nucleus through a passageway exit in the distal end region of the inserter; and
withdrawing the distal end region of the inserter from the intervertebral disc nucleus space.

52. The method of claim 51, wherein providing the disc nucleus implant instrument further comprises providing the inserter with an elongate viewing window disposed in a sidewall thereof to allow viewing of the material when in the passageway, and wherein the method further comprises positioning the material for replacing or augmenting the intervertebral disc nucleus in the passageway of the inserter adjacent to the passageway exit in the distal end region thereof prior to introducing the distal end region of the inserter into the hole in the disc annulus.

53. The method of claim 51, wherein providing the disc nucleus implant instrument further comprises providing a first post extending radially inward from at least one sidewall of the first channel member and a second post extending radially inward from at least one sidewall of the second channel member, wherein the first post and the second post are sized and positioned to facilitate loading of the material for replacing or augmenting the intervertebral disc nucleus, and wherein providing the material for replacing or augmenting an intervertebral disc nucleus comprises providing a prosthetic nucleus replacement disc including a pair of arms folded to form an inner fold when the prosthetic nucleus replacement disc is in a relaxed configuration, wherein at least two apertures are provided in the prosthetic nucleus replacement disc, each sized and positioned to receive a respective one of the first post and second post when the prosthetic nucleus replacement disc is loaded into the inserter, wherein during the transitioning of the inserter from the loading configuration to the implanting configuration, the prosthetic nucleus replacement disc disposed therein straightens to a cross-sectional size that is less than a cross-sectional size of the prosthetic nucleus replacement disc in the relaxed configuration, the straightened cross-sectional size allowing for movement of the prosthetic nucleus replacement disc through the passageway.

54. The method of claim 51, wherein providing the disc nucleus implant instrument further comprises providing a handle assembly releasably engaging a second end of the second channel member at the proximal end region of the inserter, the handle assembly comprising a pusher rod aligned with the passageway of the inserter when in the implanting configuration, and wherein the passing of the material for replacing or augmenting the intervertebral disc nucleus through the passageway exit further comprises forcing the material for replacing or augmenting the intervertebral disc nucleus from the passageway, through the passageway exit and into the intervertebral disc nucleus space employing the pusher rod of the handle assembly.
